(19) United States
(12) Patent Application Publication
(10) Pub. No.: US 2023/0135094 A1
(43) Pub. Date: May 4, 2023

(54) GARMENT

(71) Applicants: TOYOBO CO., LTD., Osaka (JP); TOYOBO STC CO., LTD., Osaka (JP)

(72) Inventors: Euichul KWON, Shiga (JP); Nobuhiro KURODA, Osaka (JP); Yuichiro OMOTE, Osaka (JP); Hideki KAWABATA, Osaka (JP)

(73) Assignees: TOYOBO CO., LTD., Osaka (JP); TOYOBO STC CO., LTD., Osaka (JP)

(21) Appl. No.: 17/911,528

(22) PCT Filed: Jan. 21, 2021
(86) PCT No.: PCT/JP2021/002057

§ 371 Date: Sep. 14, 2022

(30) Foreign Application Priority Data
Mar. 19, 2020 (JP) 2020-049716

(51) Int. Cl. A41D 13/12; A61B 5/256

(52) U.S. Cl. CPC A41D 13/1281; A61B 5/256; A41D 2500/10

(57) ABSTRACT

A garment for measuring biological information, wherein the garment is configured such that when a wearer wears the garment, a conductive member is easily disposed at a desired position on the wearer, and even when the wearer moves his/her arm or raises his/her arm, the conductive member is less likely to be displaced from the desired position on the wearer. The garment has a torso portion and a stretchable conductive member being formed on the garment with an adhesive layer interposed therebetween.

### TECHNICAL FIELD

The present invention relates to a garment for measuring biological information having a stretchable conductive member being formed on the garment with an adhesive layer interposed therebetween.

### BACKGROUND ART

In recent years, wearable garments for measuring biological information are drawing attention in a health monitoring field, a medical field, an education field, and a rehabilitation field. The wearable garments for measuring biological information are apparatuses that include a biological information measurement apparatus provided on the garment and that enable simple measurement of biological information of a person who wears it.

Various wearable garments for measuring biological information have been known so far, and the inventors of the present invention have proposed, for example, in Patent Document 1, a sensing wear that determines a measurement position enabling the most stable measurement of biological information and to which a highly closely contactable flexible electrode is attached.

### RELATED ART DOCUMENT

### Patent Documents

Patent Document 1: JP-A-2017-29692

### SUMMARY OF THE INVENTION

### Problems to Be Solved by the Invention

Such a wearable garments for measuring biological information is designed to be relatively slim in order to bring the biological information measurement apparatus thereof into close contact with the body of a wearer. Thus, when the wearer attempts to wear the wearable garment for measuring biological information, the biological information measurement apparatus is easily displaced from a desired position on the wearer, and the garment is difficult to wear. In addition, when the wearer wearing the wearable garment for measuring biological information moves his/her arm or raises his/her arm, the biological information measurement apparatus is easily displaced from the desired position, and thus it is difficult to stably and accurately measure biological information.

The present invention has been made in view of the above circumstances, and an object of the present invention is to provide a garment for measuring biological information including a stretchable conductive member formed thereon with an adhesive layer interposed therebetween, the garment for measuring biological information being configured such that: when a wearer wears the garment, the conductive member is easily disposed at a desired position on the wearer; and, even when the wearer moves his/her arm or raises his/her arm, the conductive member is less likely to be displaced from the desired position on the wearer.

### Solutions to the Problems

The garment according to the embodiment of the present invention that has solved the problem is followed.
A garment for measuring biological information having a torso portion, a stretchable conductive member being formed on the garment with an adhesive layer interposed therebetween, wherein
   the torso portion comprises at least a first clothing fabric, a second clothing fabric, and a third clothing fabric having elongation rates in a body length direction, the elongation rates being different from one another,
   the first clothing fabric is disposed in a circumferential direction around a torso,
   the conductive member is formed on the first clothing fabric,
   the second clothing fabric is disposed on each of an upper side in the body length direction and a lower side in the body length direction relative to the first clothing fabric,
   the third clothing fabric is disposed between the first clothing fabric and the second clothing fabric that is disposed on the upper side in the body length direction relative to the first clothing fabric, the third clothing fabric being disposed in each of
   a region that includes an axillary line on a right side and that occupies 5 to 30% of an entire circumference around the torso, and
   a region that includes an axillary line on a left side and that occupies 5 to 30% of the entire circumference around the torso, and
   the elongation rate in the body length direction of the third clothing fabric is higher than the elongation rate in the body length direction of the first clothing fabric.
The garment according to [1], wherein the elongation rate of the third clothing fabric at a stress of 0.5 N in the body length direction is not lower than 200% of the elongation rate of the first clothing fabric at a stress of 0.5 N in the body length direction.
The garment according to [1] or [2], wherein the third clothing fabric has a bellows shape so as to be stretchable in the body length direction.
The garment according to any one of [1] to [3], wherein the third clothing fabric is not disposed in a chest region that is located at a center position in a body width direction of a front body of the garment and that occupies 5 to 40% of the entire circumference around the torso.
The garment according to any one of [1] to [4], wherein each third clothing fabric is disposed so as to be left-right symmetric with respect to the corresponding axillary line in the circumferential direction around the torso.
The garment according to any one of [1] to [5], wherein a stress of the second clothing fabric upon elongating by 50% in a body width direction is not higher than 70% of a stress of the first clothing fabric upon elongating by 50% in the body width direction.
The garment according to any one of [1] to [6], wherein each of the first clothing fabric, the second clothing fabric, and the third clothing fabric is a knitted fabric.
The garment according to any one of [1] to [7], wherein the garment is a knitted fabric made without sewing.
The garment according to any one of [1] to [8], wherein the conductive member is an electrode configured to detect an electrical signal from a body and/or an element configured to detect a positional shift of the body.
The garment according to [9], wherein the electrode is configured to detect electrocardiographic potential or myopotential.
The garment according to any one of [1] to [10], wherein the garment is configured to cover a chest.

### Effects of the Invention

The garment of the present invention includes the torso portion. The torso portion includes at least the first clothing fabric, the second clothing fabric, and the third clothing fabric having elongation rates in the body length direction, the elongation rates being different from one another. The third clothing fabric having an elongation rate in the body length direction higher than the elongation rate in the body length direction of the first clothing fabric is disposed between the first clothing fabric on which the conductive member is formed and the second clothing fabric that is disposed on the upper side in the body length direction relative to the first clothing fabric, the third clothing fabric being disposed in each of the regions that include the respective left and right axillary lines. Consequently, when a wearer wears the garment, the second clothing fabric that is disposed on the upper side in the body length direction relative to the first clothing fabric becomes less likely to be displaced to the lower side in the body length direction. Thus, it becomes easy for the conductive member formed on the first clothing fabric to be disposed at a desired position on the wearer. In addition, the first clothing fabric becomes less likely to be displaced even when the wearer moves his/her arm or raises his/her arm. Thus, the conductive member formed on the first clothing fabric also becomes less likely to be displaced from the desired position on the wearer. As a result, biological information about the wearer can be stably and accurately measured. For example, in the case where the garment for measuring biological information is formed as a knitted fabric made without sewing and the electrode is formed by knitting a conductive yarn, the electrode is elongated following deformation of the knitted clothing fabric. Meanwhile, in the case where the electrode is formed by adhering the stretchable conductive member to the knitted clothing fabric with the adhesive layer interposed therebetween, deformation of the stretchable conductive member and deformation of the knitted clothing fabric do not necessarily occur in the same manner. Consequently, the electrode is wrinkled, and close contact between the body surface and the electrode might be hindered. On the other hand, if, as in the present invention, the second clothing fabrics are disposed such that the first clothing fabric on which the conductive member is formed is interposed between the second clothing fabrics, and the elongation rate of the third clothing fabric is set to be higher than the elongation rate of the first clothing fabric, distortion of the entire clothing fabric is absorbed by the portions at the second clothing fabrics and the third clothing fabrics. Consequently, the conductive member provided on the first clothing fabric can be kept in favorable electrical contact with the body surface.

### BRIEF DESCRIPTION OF THE DRAWINGS

FIG. 1 is a schematic view showing a state where a wearer has worn the garment of the present invention and has raised both of his/her hands.

FIG. 2 is a schematic cross-sectional view of the third clothing fabric.

### MODE FOR CARRYING OUT THE INVENTION

A garment of the present invention is a garment for measuring biological information including a stretchable conductive member formed thereon with an adhesive layer interposed therebetween.

Further, the garment includes a torso portion. The torso portion includes at least a first clothing fabric, a second clothing fabric, and a third clothing fabric having elongation rates in a body length direction, the elongation rates being different from one another. The first clothing fabric is disposed in a circumferential direction around a torso. The conductive member is formed on the first clothing fabric. The second clothing fabric is disposed on each of an upper side in the body length direction and a lower side in the body length direction relative to the first clothing fabric. The third clothing fabric is disposed between the first clothing fabric and the second clothing fabric that is disposed on the upper side in the body length direction relative to the first clothing fabric, the third clothing fabric being disposed in each of a region that includes an axillary line on a right side and that occupies 5 to 30% of an entire circumference around the torso, and a region that includes an axillary line on a left side and that occupies 5 to 30% of the entire circumference around the torso. In addition, the elongation rate in the body length direction of the third clothing fabric is higher than the elongation rate in the body length direction of the first clothing fabric. By thus disposing the third clothing fabric at a predetermined position on the torso portion of the garment, the garment becomes easy to wear, and furthermore, biological information can be stably and accurately measured.

Hereinafter, the garment of the present invention is described in detail.

The garment of the present invention is a garment for measuring biological information, and the stretchable conductive member is formed on a clothing fabric forming the garment, with the adhesive layer interposed therebetween. Since the conductive member is formed, biological information about the wearer can be measured.

Specific examples of the conductive member include: an electrode for detecting an electrical signal from the body; and an element for detecting a positional shift of the body.

The electrode for detecting an electrical signal from the body can detect an electrical signal from the body and measure biological information by bringing an electrode surface of the electrode into direct contact with the skin of the wearer. Specifically, the electrical signal acquired by the electrode is subjected to calculation and is processed by an electronic unit, whereby, for example, biological information such as an electrocardiogram, a heart rate, a pulse rate, a breathing rate, a blood pressure, a body temperature, a myopotential, or sweating is obtained. The electrode for detecting an electrical signal from the body is formed on the skin-side surface of the clothing fabric.

As the electrode, an electrode capable of detecting electrocardiographic potential or myopotential is preferable.

The electrocardiographic potential refers to a potential based on an electrical change due to cardiac movement, and an electrocardiogram can be measured on the basis of electrocardiographic potentials. The electrocardiogram means information obtained by detecting electrical changes caused by cardiac movement via an electrode on a surface of a living body and recording the changes as a waveform. The electrocardiogram is generally recorded as a waveform formed by plotting the potential difference, with the time put on the horizontal axis and the potential difference on the vertical axis. The waveform that appears every heartbeat on the electrocardiogram is mainly formed of representative five waves, i.e., a P wave, a Q wave, an R wave, an S wave, and a T wave, and there is also a U wave. A part of the start of a Q wave to the end of an S wave is sometimes called a QRS wave. An electrode capable of detecting at least the R wave among these waves is preferable. The R wave represents excitement of both the left and right ventricles and is a wave with the largest potential difference. Providing the electrode capable of detecting the R wave also enables measurement of a heart rate. That is, the time between the top of an R wave and the top of the next R wave is generally called an R-R interval (second), and the heart rate per minute can be calculated on the basis of the following formula. In the present specification, the QRS wave is to be included in the R wave unless otherwise noted. Heart rate (times / min) = 60 / R - R interval

The myopotential refers to a potential based on an electrical change due to muscle movement, and an electromyogram can be measured on the basis of myopotentials. The electromyogram refers to information obtained by detecting, via the electrode on the surface of a living body, electrical changes due to muscle movement and recording the electrical changes as a waveform. In general, the electromyogram is recorded as a waveform obtained by plotting potential differences, with the horizontal axis indicating time and the vertical axis indicating potential difference.

A specific configuration of the electrode is described later.

The element for detecting a positional shift of the body detects a physical change amount of the body and calculates a distance based on the change amount, to measure a positional shift of the body. The element for detecting a positional shift of the body may be formed on the skin-side surface of the clothing fabric or may be formed on a surface, of the clothing fabric, on an opposite side to the skin-side surface. A specific configuration of the element is described later.

On the garment of the present invention, both the electrode for detecting an electrical signal from the body and the element for detecting a positional shift of the body may be formed, or either the electrode for detecting an electrical signal from the body or the element for detecting a positional shift of the body may be formed.

The above conductive member is required to have stretchability. The stretchability makes it less likely for the conductive member to be displaced from a desired position on the wearer even when the wearer moves his/her arm or raises his/her arm.

The stretchable conductive member is formed on the clothing fabric forming the garment, with the adhesive layer interposed therebetween.

Hereinafter, an exemplary embodiment of the garment of the present invention including the clothing fabric which forms the garment and on which the stretchable conductive member is formed with the adhesive layer interposed therebetween, is described with reference to the drawings. However, the present invention is not limited by the drawings presented below and, as a matter of course, can be carried out with modifications made within a scope of a gist described above and below, and any of these modifications is included in the technical scope of the present invention.

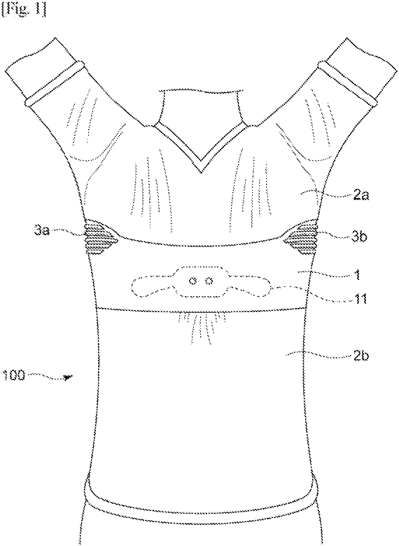

FIG. 1 is a schematic view showing a state where a wearer has worn the garment of the present invention and has raised both of his/her hands, and is a front view of the wearer. In FIG. 1, the up-down direction is defined as a body length direction, the left-right direction is defined as a body width direction, the right hand side of the wearer is defined as a right side, and the left hand side of the wearer is defined as a left side.

A garment 100 of the present invention includes a torso portion. The torso portion includes at least a first clothing fabric, second clothing fabrics, and third clothing fabrics having elongation rates in the body length direction, the elongation rates being different from one another. The elongation rates in the body length direction can be measured according to method A (constant-speed elongation method) stipulated in 8.16.1 of JIS L 1096. A grip interval for a test piece only has to be set to, for example, 3 cm × 3 cm, and a tension speed only has to be set to, for example, 30 cm/min.

Among the first clothing fabric, the second clothing fabrics, and the third clothing fabrics of the garment of the present invention, the first clothing fabric 1 is disposed in a circumferential direction around the torso (100% of the entire circumference around the torso). Each of conductive members 11 is formed on the first clothing fabric 1 with an adhesive layer interposed therebetween. The first clothing fabric 1 is fitted to the body shape of a wearer such that the conductive member 11 comes into close contact with the body of the wearer. In FIG. 1, the conductive member 11 is formed on the skin-side surface of the first clothing fabric 1, and a region in which the conductive member 11 is formed is indicated by a broken line.

The first clothing fabric 1 is disposed in a region that preferably occupies not lower than 80%, more preferably not lower than 90%, further preferably not lower than 95%, and most preferably 100% of the entire circumference around the torso.

The weight per unit area of the first clothing fabric is preferably 100 to 300 g/m². If the weight per unit area of the first clothing fabric is not lower than 100 g/m², the strength of the clothing fabric is easily increased. In addition, if the weight per unit area of the clothing fabric is excessively low, translucency is imparted to the clothing fabric, and thus excessively low weights per unit area tend to be avoided particularly if the garment is an undershirt. The weight per unit area of the first clothing fabric is more preferably not lower than 130 g/m² and further preferably not lower than 150 g/m². On the other hand, if the weight per unit area of the first clothing fabric is not higher than 300 g/m², the weight of the clothing fabric can be made easy to reduce. In addition, if the weight per unit area of the clothing fabric is excessively high, the excessively high weight per unit area causes the garment to look thick when worn, and thus excessively high weights per unit area tend to be avoided particularly if the garment is an undershirt. The weight per unit area of the first clothing fabric is more preferably not higher than 280 g/m² and further preferably not higher than 250 g/m². The weight per unit area of the clothing fabric can be measured according to a method described in EXAMPLES described later (the same applies below).

In the garment of the present invention, a second clothing fabric 2a is disposed on the upper side in the body length direction relative to the first clothing fabric 1, and a second clothing fabric 2b is disposed on the lower side in the body length direction relative to the first clothing fabric 1.

A stress of the second clothing fabric 2a upon elongating by 50% in the body width direction and a stress of the second clothing fabric 2b upon elongating by 50% in the body width direction are preferably not higher than 70% of a stress of the first clothing fabric 1 upon elongating by 50% in the body width direction. If the second clothing fabrics 2a and 2b which are less tight than the first clothing fabric 1 are disposed on the upper side in the body length direction and the lower side in the body length direction relative to the first clothing fabric 1, the sense of excessive tightness at the time of wearing can be mitigated, and the conductive member formed on the first clothing fabric 1 becomes less likely to be displaced from the desired position so that biological information about the wearer can be stably and accurately measured. The stress of the second clothing fabric 2a upon elongating by 50% in the body width direction and the stress of the second clothing fabric 2b upon elongating by 50% in the body width direction are more preferably not higher than 60% and further preferably not higher than 50% of the stress of the first clothing fabric 1 upon elongating by 50% in the body width direction. Regarding lower limits of the stress of the second clothing fabric 2a upon elongating by 50% in the body width direction and the stress of the second clothing fabric 2b upon elongating by 50% in the body width direction, the stresses are, for example, preferably not lower than 10%, more preferably not lower than 20%, and further preferably not lower than 25% of the stress of the first clothing fabric 1 upon elongating by 50% in the body width direction. If the stress of the second clothing fabric 2a upon elongating by 50% in the body width direction and the stress of the second clothing fabric 2b upon elongating by 50% in the body width direction are different from each other, the higher one of the stresses is preferably not higher than 70% of the elongation rate in the body width direction of the first clothing fabric 1.

The stress of the second clothing fabric 2a upon elongating by 50% in the body width direction and the stress of the second clothing fabric 2b upon elongating by 50% in the body width direction may be different from each other, but are preferably equal to each other.

The stresses of the second clothing fabrics 2a and 2b upon elongating by 50% in the body width direction can be measured according to method A (constant-speed elongation method) stipulated in 8.16.1 of JIS L 1096. A grip interval for a test piece only has to be set to, for example, 3 cm × 3 cm, and a tension speed only has to be set to, for example, 30 cm/min.

The weight per unit area of each of the second clothing fabrics is preferably 80 to 250 g/m². If the weight per unit area of the second clothing fabric is not lower than 80 g/m², the strength of the clothing fabric is more likely to be increased. In addition, if the weight per unit area of the clothing fabric is excessively low, translucency is imparted to the clothing fabric, and thus excessively low weights per unit area tend to be avoided particularly if the garment is an undershirt. The weight per unit area of the second clothing fabric is more preferably not lower than 100 g/m² and further preferably not lower than 130 g/m². On the other hand, if the weight per unit area of the second clothing fabric is not higher than 250 g/m², the weight of the clothing fabric can be made easy to reduce. In addition, if the weight per unit area of the clothing fabric is excessively high, the excessively high weight per unit area causes the garment to look thick when worn, and thus excessively high weights per unit area tend to be avoided particularly if the garment is an undershirt. The weight per unit area of the second clothing fabric is more preferably not higher than 230 g/m² and further preferably not higher than 200 g/m².

In the garment of the present invention, a third clothing fabric 3a is disposed between the first clothing fabric 1 and the second clothing fabric 2a disposed on the upper side in the body length direction relative to the first clothing fabric 1, the third clothing fabric 3a being disposed in a region that includes the axillary line on the right side and that occupies 5 to 30% of the entire circumference around the torso. In addition, a third clothing fabric 3b is disposed between the first clothing fabric 1 and the second clothing fabric 2a disposed on the upper side in the body length direction relative to the first clothing fabric 1, the third clothing fabric 3b being disposed on a region that includes the axillary line on the left side and that occupies 5 to 30% of the entire circumference around the torso. An example of the form of each of the third clothing fabrics 3a and 3b is described with reference to the drawings. The present invention is not limited to the drawings.

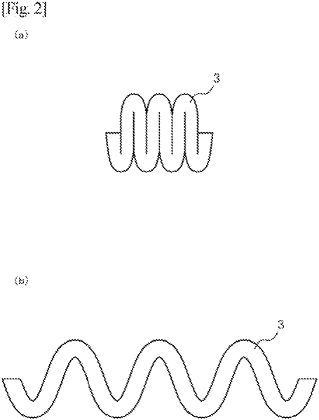

FIG. 2 is a schematic cross-sectional view of the third clothing fabric 3. In the drawing, the left-right direction corresponds to the body length direction. FIG. 2(a) shows a state where the third clothing fabric 3 has not been elongated in the body length direction. FIG. 2(b) shows a state where the third clothing fabric 3 has been elongated in the body length direction.

Since the third clothing fabrics 3a and 3b are disposed between the first clothing fabric 1 and the second clothing fabric 2a disposed on the upper side in the body length direction relative to the first clothing fabric 1, the third clothing fabrics 3a and 3b are elongated in the body length direction as shown in FIG. 2(b) when the wearer wears the garment. Consequently, the second clothing fabric 2a disposed on the upper side in the body length direction relative to the first clothing fabric 1 becomes less likely to be displaced to the lower side in the body length direction. Thus, it becomes easy for the conductive member formed on the first clothing fabric 1 to be disposed at the desired position on the wearer. In addition, when the wearer moves his/her arm or raises his/her arm so that the second clothing fabric 2a is pulled, the third clothing fabrics 3a and 3b are elongated in the body length direction as shown in FIG. 2(b), and thus a buffer effect of the third clothing fabrics 3a and 3b makes it less likely for the first clothing fabric 1 to be displaced from a desired position. Therefore, the conductive member formed on the first clothing fabric 1 also becomes less likely to be displaced from the desired position on the wearer. As a result, biological information about the wearer can be stably and accurately measured.

The shape of each of the third clothing fabrics 3a and 3b is not particularly limited, and examples of the shape include a bellows shape, a net shape, and the like that allows the third clothing fabric to be elongated in the body length direction. The shape is preferably a bellows shape. If a clothing fabric is provided with projections and recesses in the structure thereof as is the case with a bellows shape or a net shape, the structure with the projections and the recesses are elongated prior to elongating of yarns when the clothing fabric is elongated. Consequently, the stress that is applied upon the elongating can be made very small. Therefore, the stress is made less likely to be applied to the first clothing fabric 1, whereby the first clothing fabric 1 can be prevented from being displaced from the skin.

An example of a bellows structure is described as follows. For example, in the case where each of the third clothing fabrics 3a and 3b is a circular knitted fabric, the bellows structure can be obtained by setting, as the up-down direction of a body-contact clothing fabric, the course direction of a knitted fabric such as a circular rib fabric, a fabric knitted with needles drawn off from the front and back sides thereof (hereinafter, draw-off fabric), or a rib knitted fabric. In addition, if cylinder needles and dial needles are alternately drawn off at an interval of one needle or several needles in the course direction so as to form a circular rib fabric structure or a draw-off fabric structure, a larger projection-and-recess structure can be obtained. Meanwhile, in the case where, for example, the third clothing fabrics 3a and 3b are weft knitted fabrics, a garter structure composed of a plurality of face stitches and a plurality of back stitches arranged in the wale direction is defined as one unit which is repeated so that the bellows structure can be obtained. In the bellows structure as seen from the front surface side thereof, consecutive face stitches in the wale direction form a portion bent backward, and consecutive back stitches in the wale direction form a raised portion. In the repeating unit, the number of the face stitches is preferably set to 3 to 20, and the number of the back stitches is preferably set to 3 to 20.

The third clothing fabric 3a only has to be disposed in a region that extends in a direction around the torso and that includes the axillary line on the right side. Thus, the third clothing fabric 3a may be disposed only on a front body including the axillary line on the right side, or may be disposed only on a back body including the axillary line on the right side. However, the third clothing fabric 3a is preferably disposed so as to be left-right symmetric with respect to the axillary line in the circumferential direction around the torso. The third clothing fabric 3a is disposed in a region that preferably occupies not lower than 10% and more preferably occupies not lower than 15% of the entire circumference around the torso. Meanwhile, the third clothing fabric 3a is disposed in a region that preferably occupies not higher than 25% and more preferably occupies not higher than 20% of the entire circumference around the torso.

The third clothing fabric 3b only has to be disposed in a region that extends in the direction around the torso and that includes the axillary line on the left side. Thus, the third clothing fabric 3b may be disposed only on the front body including the axillary line on the left side, or may be disposed only on the back body including the axillary line on the left side. However, the third clothing fabric 3b is preferably disposed so as to be left-right symmetric with respect to the axillary line in the circumferential direction around the torso. The third clothing fabric 3b is disposed in a region that preferably occupies not lower than 10% and more preferably occupies not lower than 15% of the entire circumference around the torso. Meanwhile, the third clothing fabric 3b is disposed in a region that preferably occupies not higher than 25% and more preferably occupies not higher than 20% of the entire circumference around the torso.

A length of the region in which the third clothing fabric 3a is disposed and a length of the region in which the third clothing fabric 3b is disposed may be different from each other in the direction around the torso. However, the lengths are preferably equal to each other in order to attain balance between the left and right sides.

The elongation rate of the third clothing fabric 3a at a stress of 0.5 N in the body length direction and the elongation rate of the third clothing fabric 3b at a stress of 0.5 N in the body length direction are preferably not lower than 200% of the elongation rate of the first clothing fabric 1 at a stress of 0.5 N in the body length direction. Since the third clothing fabrics 3a and 3b which are more easily elongated than the first clothing fabric 1 are disposed between the first clothing fabric 1 and the second clothing fabric 2a disposed on the upper side in the body length direction relative to the first clothing fabric 1, the third clothing fabrics 3a and 3b are elongated when the wearer wears the garment. Consequently, the second clothing fabric 2a disposed on the upper side in the body length direction relative to the first clothing fabric 1 becomes less likely to be displaced to the lower side in the body length direction. Thus, it becomes easy for the conductive member formed on the first clothing fabric 1 to be disposed at the desired position on the wearer. In addition, even when the wearer moves his/her arm or raises his/her arm so that the second clothing fabric 2a is pulled, the buffer effect of the third clothing fabrics 3a and 3b makes it less likely for the first clothing fabric 1 to be displaced from the desired position. Therefore, the conductive member formed on the first clothing fabric 1 also becomes less likely to be displaced from the desired position on the wearer. As a result, biological information about the wearer can be stably and accurately measured. The elongation rate of the third clothing fabric 3a at a stress of 0.5 N in the body length direction and the elongation rate of the third clothing fabric 3b at a stress of 0.5 N in the body length direction are more preferably not lower than 300% and further preferably not lower than 400% of the elongation rate in the body length direction of the first clothing fabric 1. Regarding upper limits of the elongation rate of the third clothing fabric 3a at a stress of 0.5 N in the body length direction and the elongation rate of the third clothing fabric 3b at a stress of 0.5 N in the body length direction, the elongation rates are, for example, preferably not higher than 1000%, more preferably not higher than 800%, and further preferably not higher than 600% of the elongation rate of the first clothing fabric 1 at a stress of 0.5 N in the body length direction. If the elongation rate of the third clothing fabric 3a at a stress of 0.5 N in the body length direction and the elongation rate of the third clothing fabric 3b at a stress of 0.5 N in the body length direction are different from each other, the lower one of the elongation rates is preferably not lower than 200% of the elongation rate of the first clothing fabric 1 at a stress of 0.5 N in the body length direction.

The elongation rate of the third clothing fabric 3a at a stress of 0.5 N in the body length direction and the elongation rate of the third clothing fabric 3b at a stress of 0.5 N in the body length direction may be different from each other. However, the elongation rates are preferably equal to each other.

Each of the third clothing fabrics may be formed as a part of the first clothing fabric. That is, the third clothing fabric having an elongation rate in the body length direction higher than the elongation rate in the body length direction of the first clothing fabric may be disposed in the region of the first clothing fabric.

The weight per unit area of the third clothing fabric is not particularly limited and is difficult to define since the weight per unit area differs depending on the manner of collecting the third clothing fabric. However, the weight per unit area is preferably about 200 to 400 g/m². The weight per unit area of the third clothing fabric is more preferably not lower than 230 g/m² and further preferably not lower than 250 g/m². On the other hand, the weight per unit area of the third clothing fabric is more preferably not higher than 380 g/m² and further preferably not higher than 350 g/m².

It is preferable that the third clothing fabric is not disposed in a chest region that is located at a center position in the body width direction of the front body of the garment and that occupies 5 to 40% of the entire circumference around the torso in the body width direction. If the third clothing fabric is not disposed in this region, it becomes easy for the conductive member formed on the first clothing fabric to be disposed at the desired position on the wearer when the wearer wears the garment. As a result, biological information about the wearer can be stably and accurately measured. The region in which the third clothing fabric is not disposed, more preferably occupies not lower than 10% and further preferably occupies not lower than 20% of the entire circumference around the torso in the body width direction. Meanwhile, the region more preferably occupies not higher than 38% and further preferably occupies not higher than 35% of the entire circumference around the torso in the body width direction.

The forms of the first clothing fabric, each second clothing fabric, and each third clothing fabric are not particularly limited as long as each of the clothing fabrics is in the form of a fabric. The clothing fabrics may each be in the form of either a knitted fabric or a woven fabric. All of the first clothing fabric, the second clothing fabric, and the third clothing fabric are more preferably in the form of knitted fabrics. Each of the knitted fabrics is preferably a weft knitted fabric or a warp knitted fabric and more preferably a weft knitted fabric. Examples of the weft knitted fabric also include circular knitted fabrics.

Examples of the weft knitted fabric (circular knitted fabric) include fabrics having stitch structures such as jersey stitch (plain stitch), bare jersey stitch, welt jersey stitch, circular rib stitch (rib stitch), purl stitch, half tubular stitch, interlock stitch, tuck stitch, float stitch, half cardigan stitch, lace stitch, plated stitch. Among these stitches, the jersey stitch, the circular rib stitch, or the interlock stitch is preferable, and the jersey stitch or the interlock stitch is more preferable. Each of these stitch structures is a structure with at least one flat surface, and thus enables increase in the peel strength of the electrode with respect to the clothing fabric. Examples of the warp knitted fabric include fabrics having stitch structures such as single denbigh stitch, open-loop denbigh stitch, single atlas stitch, double cord stitch, half stitch, half base stitch, satin stitch, tricot stitch, half tricot stitch, raschel stitch, and jacquard stitch.

Examples of the woven fabric include fabrics having woven structures such as plain weave, twill weave (twill), satin weave, multi-weave, dobby weave, jacquard weave. The woven fabric may be made as a patterned cloth such as a striped cloth or a checked cloth by using a plurality of types of yarns dyed in advance in different colors, or may be made as a patterned woven cloth by using a jacquard loom. In the case where the clothing fabric is used for a garment such as a shirt fabric or a blouse fabric, the plain weave or the twill weave (twill) is particularly preferable. In order to increase the peel strength between the electrode and the clothing fabric, a structure with less projections and recesses from the skin-side surface and less floating portions of yarns is favorable, and thus the plain weave is more preferable.

The garment of the present invention may be made by sewing at least the first clothing fabric, the second clothing fabric, and the third clothing fabric. However, the garment is preferably made without sewing at least the first clothing fabric, the second clothing fabric, and the third clothing fabric, and the entire garment may be made without sewing.

Each of the knitted fabric and the woven fabric is preferably a fabric containing at least one type of fiber selected from the group consisting of natural fiber, synthetic fiber, regenerated fiber, and semi-synthetic fiber. Examples of the natural fiber include cotton, hemp, wool, silk, and the like. Among these natural fibers, cotton is preferable. If the fabric contains cotton, the hygroscopicity, the water absorbency, the heat retaining properties, and the like of the fabric are improved. The natural fiber may be used as is or may be subjected to post-treatment such as hydrophilization treatment or anti-stain treatment. Examples of the synthetic fiber include: acrylic; polyesters such as polyethylene terephthalate, polyethylene naphthalate, polybutylene terephthalate, polytrimethylene terephthalate, polyethylene isophthalate, polylactic acid, and polyacrylate; polyamides such as nylon 6 and nylon 66; and the like. Examples of the regenerated fiber include: rayons such as modal; cupra; polynosic; lyocell; and the like. Examples of the semi-synthetic fiber include acetate, triacetate, and the like. These types of fibers may be used singly, or two or more of these types of fibers may be used in combination.

The garment of the present invention preferably covers, for example, a chest. Specific examples of the garment include a T-shirt, a polo shirt, a camisole, sports innerwear, a hospital gown, and pajamas.

Next, a specific configuration of the conductive member formed on the first clothing fabric is described in detail.

As described above, specific examples of the conductive member include: an electrode for detecting an electrical signal from the body; and an element for detecting a positional shift of the body.

The electrode may be used as an electric contact such as a connector in order to detect an electrical signal (bioelectric potential) from the body mainly through contact with the skin, or may be used as a detection terminal of a contactless proximity sensor.

The electrode has stretchability so as to be able to follow motion of the wearer (subject being measured).

The electrode preferably has the shape of a sheet. If the electrode has the shape of a sheet, an electrode surface thereof can be made wide, and thus a contact area with the skin of the wearer can be ensured. The electrode having the shape of a sheet preferably has stretchability, and preferably furthermore, bendability. The area of the electrode surface is preferably 5 to 100 cm². The average thickness of the electrode is preferably 10 to 500 µm. The shape of the electrode is preferably such that: the electrode is fitted along a curve, of the body, that corresponds to the position at which the electrode is disposed; and the electrode easily follows movement of the body to come into close contact with the body. Examples of the shape include a tetragon, a triangle, a five or more sided polygon, a circle, an ellipse, and the like. If the electrode has the shape of a polygon, the vertices of the polygon may be rounded so as not to injure the skin.

The electrode preferably includes: an insulating layer formed on the skin-side surface of the clothing fabric; and a conductive layer formed on the insulating layer. If the insulating layer is provided on the garment side of the conductive layer, the insulating layer suppresses elongation of the clothing fabric, whereby the conductive layer can be prevented from being excessively elongated.

The insulating layer only has to be a layer acting as an insulator and may act as, in addition to an insulator, a water stoppage layer. The water stoppage layer prevents water from reaching the conductive layer, the water coming from an opposite side, of the clothing fabric worn, to the side on which the insulating layer is stacked. That is, the opposite side is the outer side of the garment. In addition, the insulating layer may have adhesiveness.

The insulating layer may be directly formed on the skin-side surface of the clothing fabric. Alternatively, the insulating layer may be fixed by adhesion to the skin-side surface of the clothing fabric with an adhesive layer (described later) interposed therebetween.

The insulating layer may be formed of insulating resin. As the insulating resin, it is possible to preferably use, for example, polyurethane-based resin, silicone-based resin, vinyl chloride-based resin, epoxy-based resin, and polyester elastomer. Among these types of resin, polyurethane-based resin is more preferable, and has further good adhesiveness to the conductive layer. A single type of resin, or two or more types of resin may be used as the resin forming the insulating layer.

The method for forming the insulating layer is not particularly limited, and examples of the method include a method including: dissolving or dispersing the insulating resin in a solvent (preferably, water); applying the resulting solution on, or performing printing with the resulting solution on, a release paper or a release film to form a coating film; and volatilizing the solvent contained in the coating film to dry the coating film. A commercially available insulating resin sheet or insulating resin film can also be used.

The average film thickness of the insulating layer is preferably 10 to 200 µm. If the average film thickness of the insulating layer is not smaller than 10 µm, it becomes easy to exhibit an insulating effect and a stretch preventing effect for the clothing fabric. The average film thickness of the insulating layer is more preferably not smaller than 30 µm and further preferably not smaller than 40 µm. On the other hand, if the average film thickness of the insulating layer is not larger than 200 µm, the stretchability thereof is improved. The average film thickness of the insulating layer is more preferably not larger than 180 µm and further preferably not larger than 150 µm.

Alternatively, the conductive layer may be formed on the skin-side surface of the clothing fabric without the insulating layer interposed therebetween but with the adhesive layer interposed therebetween.

The conductive layer only has to be able to detect and transmit an electrical signal (bioelectric potential) from the body.

A conductive layer containing a conductive filler and a resin is preferable, a conductive layer containing a conductive filler and a stretchable resin is more preferable, and a conductive layer containing a conductive filler and elastomer is further preferable. These conductive layers can each be formed by using a composition (hereinafter, sometimes referred to as a conductive paste) obtained by dissolving or dispersing the components in an organic solvent.

As the conductive filler, it is possible to use, for example, a metal powder, metal nanoparticles, and a conductive material other than the metal powder. A single type of material, or two or more types of materials may be used as the conductive filler.

Examples of the metal powder include noble metal powders such as a silver powder, a gold powder, a platinum powder, and a palladium powder; base metal powders such as a copper powder, a nickel powder, an aluminum powder, and a brass powder; a plated powder obtained by plating different types of particles made of inorganic substances such as a base metal and silica with a noble metal such as silver; and an alloyed base metal powder obtained by alloying a base metal and a noble metal such as silver. Among these materials, a silver powder and/or a copper is preferable and enables the conductive layer to exhibit high conductivity at low costs.

Examples of the metal nanoparticles include particles having a particle size of several to several tens of nanometers among the metal powders described above.

Examples of the conductive material other than the metal powder include carbon-based materials such as graphite, carbon black, and carbon nanotube. The conductive material other than the metal power preferably has a mercapto group, an amino group, or a nitrile group on a surface thereof, or preferably has a surface thereof surface-treated with rubber containing a sulfide bond and/or a nitrile group.

The conductive layer may be a single layer. Alternatively, the conductive layer may be obtained by stacking or arraying two or more types of conductive layers among which the type of the conductive filler, the amount of the conductive filler added, or the like differs, so that the plurality of conductive layers are integrated with one another.

The conductive filler content of the conductive layer (in other words, the conductive filler content with respect to the total solid content of the conductive paste for forming the conductive layer) is preferably not lower than 25 mass%. If the conductive filler content is not lower than 25 mass%, the conductivity of the conductive layer is improved. The conductive filler content is more preferably not lower than 40 mass% and further preferably not lower than 60 mass%. Meanwhile, the conductive filler content of the conductive layer is preferably not higher than 98 mass%. If the conductive filler content is not higher than 98 mass%, the stretchability of the conductive layer can be improved, and cracks or the like become less likely to occur when the electrode or the like is elongated. The conductive filler content is more preferably not higher than 95 mass% and further preferably not higher than 90 mass%.

The stretchable resin that is contained in the conductive layer preferably contains, for example, a rubber containing a sulfur atom and/or a rubber containing a nitrile group. The sulfur atom and the nitrile group have high affinity for the conductive filler (particularly, the metal powder), and the rubber has high stretchability, whereby generation of cracks or the like can be made easily avoidable even upon elongating.

As the rubber containing a sulfur atom, elastomer may be used besides the rubber containing a sulfur atom. The sulfur atom is contained in the form of a sulfide bond or a disulfide bond in the main chain of a polymer, a mercapto group in a side chain or at a terminal, or the like.

Examples of the rubber containing a sulfur atom include polysulfide rubber, polyether rubber, polyacrylate rubber, and silicone rubber that contain a mercapto group, a sulfide bond, or a disulfide bond. Particularly, polysulfide rubber, polyether rubber, polyacrylate rubber, and silicone rubber that contain a mercapto group are preferable. The rubber containing a sulfur atom preferably has a content of the sulfur atom of 10 to 30 mass%.

As the rubber containing a nitrile group, elastomer may be used besides the rubber containing a nitrile group. Particularly, an acrylonitrile-butadiene copolymer rubber that is a copolymer of butadiene and acrylonitrile is a preferable example. As a commercially available product that can be used as the rubber containing a nitrile group, preferable examples are Nipol (registered trademark) 1042 and Nipol (registered trademark) DN003 that are manufactured by Zeon Corporation. The rubber containing a nitrile group has an amount of the nitrile group (particularly, the acrylonitrile-butadiene copolymer rubber has an amount of acrylonitrile) of preferably 18 to 50 mass%, more preferably 20 to 45 mass%. If the bonded acrylonitrile content of the acrylonitrile-butadiene copolymer rubber is not higher than 50 mass%, the rubber elasticity can be improved. On the other hand, if the bonded acrylonitrile content of the acrylonitrile-butadiene copolymer rubber is not lower than 18 mass%, the affinity for the conductive filler (particularly, the metal powder) is improved.

The total amount of the rubber containing a sulfur atom and the rubber containing a nitrile group in 100 mass% of the stretchable resin that is contained in the conductive layer, is preferably not lower than 95 mass%, more preferably not lower than 98 mass%, and further preferably not lower than 99 mass%.

The resin content of the conductive layer (in other words, the resin solid content with respect to the total solid content of the conductive paste for forming the conductive layer) is preferably not lower than 2 mass% and not higher than 75 mass%. The resin content is more preferably not lower than 5 mass% and further preferably not lower than 10 mass%. Meanwhile, the resin content is more preferably not higher than 50 mass% and further preferably not higher than 40 mass%.

The conductive layer can be formed directly on the insulating layer, using a composition (conductive paste) obtained by dissolving or dispersing the components described above in an organic solvent, or can be formed by forming a coating film in a desired pattern through coating or printing, and volatilizing the organic solvent included in the coating film and thus drying the coating film. The conductive layer may also be formed by applying the conductive paste to or performing printing with the conductive paste on a release sheet or the like to form a coating film, volatilizing the organic solvent included in the coating film and thus drying the coating film to form a sheet-shaped conductive layer in advance, and stacking the conductive layer in a desired pattern on the insulating layer. The conductive paste may be prepared employing a conventionally known method for dispersing a powder substance in a liquid, and can be prepared by uniformly dispersing the conductive filler in the stretchable resin. The conductive paste may be prepared by mixing, for example, the metal powder, the metal nanoparticles, or the conductive material other than the metal powder with a resin solution, and then uniformly dispersing the contents by an ultrasonic method or with a mixer, a triple ball mill, or a ball mill. A plurality of these types of means can be used in combination. The method for applying the conductive paste or performing printing with the conductive paste is not particularly limited, and a printing method can be employed, such as coating, screen printing, litho offset printing, ink-jet printing, flexo printing, gravure printing, gravure offset printing, stamping, dispensing, or squeegee printing.

The conductive layer has a dried film thickness of preferably 10 to 150 µm. The conductive layer having a dried film thickness of 10 µm or more makes the electrode less likely to be deteriorated even subjected to repetitive stretch and contraction. The conductive layer has a dried film thickness of more preferably not smaller than 20 µm, further preferably not smaller than 30 µm. On the other hand, the conductive layer having a dried film thickness of 150 µm or less improves the stretchability and thus facilitate improvement of wear comfort. The conductive layer has a dried film thickness of more preferably not larger than 130 µm, further preferably not larger than 100 µm.

The electrode may be a sheet-shaped electrode having a conductive structure. Examples of the electrode having a conductive structure include: woven fabrics, knitted fabrics, and nonwoven fabrics made of a conductive fiber or a conductive yarn obtained by covering a base fiber with a conductive polymer; woven fabrics, knitted fabrics, and nonwoven fabrics made of a fiber having a surface covered with a conductive metal such as silver, gold, copper, or nickel; woven fabrics, knitted fabrics, and nonwoven fabrics made of a conductive yarn made of filaments of the conductive metal; woven fabrics, knitted fabrics, and nonwoven fabrics made of a conductive yarn obtained by mixed spinning of filaments of the conductive metal and a non-conductive fiber; woven fabrics, knitted fabrics, and nonwoven fabrics made of other fibers or yarns; fabrics obtained by embroidering a non-conductive fabric with any of these conductive yarns; and the like. These conductive structures may be fixed by adhesion to the skin-side surface of the clothing fabric with the adhesive layer (described later) interposed therebetween.

The electrode is preferably provided on a chest part or an abdominal part under the chest part of the garment. The electrode provided on the chest part or the abdominal part under the chest part of the garment enables accurate measurement of biological information. The electrode is more preferably provided in a region, of the garment, that is brought into contact with the skin between the upper end of the seventh rib and the lower end of the ninth rib of the wearer. The electrode is preferably provided in a region, of the garment, that faces the ventral side of the wearer and that is interposed between lines, the lines being parallel with the left and right posterior axillary lines of the wearer and being drawn at positions 10 cm away from the posterior axillary lines of the wearer toward the back of the wearer. The electrode is preferably provided in the form of an arc along the torso of the wearer.

The number of the electrodes to be provided on the garment is at least two. The two electrodes are preferably provided on the chest part or the abdominal part under the chest part of the garment, and the two electrodes are preferably provided in the region that faces the ventral side of the wearer and that is interposed between lines, the lines being parallel with the left and right posterior axillary lines of the wearer and being drawn at positions 10 cm away from the posterior axillary lines of the wearer toward the back of the wearer. In the case where three or more electrodes are provided, the positions at which the third and subsequent electrodes are provided are not particularly limited. The third and subsequent electrodes may be provided on, for example, the back body of the clothing fabric.

The garment preferably includes the electrodes and a wire connected to the electrodes. The wire can make connection between an electrode and an electronic unit having a function of performing calculation on an electrical signal acquired by the electrode, and the like. The wire preferably includes: a first insulating layer formed on the skin-side surface of the clothing fabric; a conductive layer formed on the skin-side surface of the first insulating layer; and a second insulating layer formed on the skin-side surface of the conductive layer. For the features of the first insulating layer, descriptions of the above insulating layer of the electrode can be referred to. The first insulating layer and the above insulating layer of the electrode are preferably formed from the same material so as to be integrated with each other. Likewise, the conductive layer of the wire is preferably formed from the same material as that of the above conductive layer of the electrode so as to be integrated with the above conductive layer.

The wire described above preferably includes a second insulating layer formed on the conductive layer. The second insulating layer provided can prevent the conductive layer from contacting with, for example, water such as rain, snow, and sweat. Examples of resin forming the second insulating layer include the same types of resin as the above-described resin forming the first insulating layer, and preferably used resin is also the same. A single type of resin, or two or more types of resin may also be used as the resin forming the second insulating layer. The resin forming the second insulating layer may be the same as or different from the resin forming the first insulating layer, but is preferably the same. The use of the same resin can reduce damage on the conductive layer caused by bias in coverage of the conductive layer and in stress during stretch or contraction of the wire. The second insulating layer can be formed by the same forming method as the first insulating layer. A commercially available resin sheet or resin film can also be used.

The average film thickness of the second insulating layer is preferably 10 to 200 µm. If the average film thickness of the second insulating layer is not smaller than 10 µm, the insulating effect and the stretch preventing effect are easily exhibited. The average film thickness of the second insulating layer is more preferably not smaller than 30 µm and further preferably not smaller than 40 µm. On the other hand, if the average film thickness of the second insulating layer is not larger than 200 µm, the stretchability thereof is improved. The average film thickness of the second insulating layer is more preferably not larger than 180 µm and further preferably not larger than 150 µm.

As the wire, a conductive fiber or a conductive yarn may be used. As the conductive fiber or the conductive yarn, it is possible to use, for example, one obtained by plating a surface of an insulating fiber with a metal, one obtained by twisting a thin metal wire in a yarn, one obtained by impregnating the intervals between fibers such as microfibers with a conductive polymer, or the thin metal wire.

The average thickness of the wire is preferably 10 to 500 µm. If the thickness of the wire is excessively small, the conductivity thereof might become insufficient. The average thickness of the wire is more preferably not smaller than 30 µm and further preferably not smaller than 50 µm. Meanwhile, the wire having an excessively large thickness gives the wearer the feeling of a foreign body and thus gives the wearer discomfort. Considering this, the average thickness of the wire is more preferably not larger than 300 µm and further preferably not larger than 200 µm.

The shape of the wire is not particularly limited and may be a straight line, a curve, or a geometrical pattern. Examples of the geometrical pattern include a zigzag pattern, a continuous horseshoe pattern, a wave pattern, and the like. The electrode having a geometrical pattern can be formed by using, for example, metal foil. The conductive fiber or the conductive yarn as the wire may be fixed to the clothing fabric through embroidery or the like.

A method for forming the electrode and the wire on the clothing fabric is not particularly limited as long as the method does not lead to reduction in the stretchabilities of the electrode and the wire, and examples of the method include methods such as a method involving stacking with an adhesive layer interposed and a method involving stacking through heat pressing.

Examples of a method for applying an adhesive for forming the adhesive layer include a method involving powder application, spray application, coating, printing, or adhesive sheet pasting, followed by heat treatment, pressure bonding, or the like.

As the adhesive, it is possible to use, for example, a urea resin-based adhesive, a melamine resin-based adhesive, a phenol resin-based adhesive, a solvent type adhesive, an aqueous type adhesive, a reactive type adhesive, a hot-melt adhesive, or the like. These types of adhesives may be used singly, or two or more of these types of adhesives may be used in combination. Among these adhesives, a hot-melt adhesive is preferable. As the solvent type adhesive, it is possible to use, for example, a vinyl acetate resin-based solvent type adhesive, a rubber-based solvent type adhesive, another resin-based solvent type adhesive, or the like. As the aqueous type adhesive, it is possible to use, for example, an EVA resin-based emulsion type adhesive, an acrylic resin-based emulsion type adhesive, a vinyl acetate resin-based emulsion type adhesive, a vinyl acetate copolymer resin-based emulsion type adhesive, or the like. As the reactive type adhesive, it is possible to use, for example, an epoxy resin-based adhesive, a cyanoacrylate-based adhesive, a polyurethane-based adhesive, an acrylic resin-based adhesive, or the like. As the hot-melt adhesive, it is possible to use, for example, a polyethylene-based adhesive, a polyamide-based adhesive, a soft polyvinyl chloride-based adhesive, a polyvinyl acetate-based adhesive, a polyester-based adhesive, a polyurethane-based adhesive, or the like. Among these adhesives, a polyurethane-based adhesive is preferable since a polyurethane-based adhesive has a high flexibility and allows the flexibility of a peripheral portion of the electrode to be kept high after adhesion. Usage of a thermoplastic polyurethane-based adhesive is more preferable. As the hot-melt adhesive, it is possible to use a hot-melt adhesive in any of various forms such as the forms of a sheet, powder, and liquid. Among these hot-melt adhesives, a hot-melt adhesive in the form of a sheet is preferable since such a hot-melt adhesive makes it easy to improve the peel strength of the electrode with respect to the clothing fabric. The insulating layer and the adhesive layer may be made from the same material.

The garment preferably includes an electronic unit or the like having a function of performing calculation on an electrical signal acquired by the electrode. If the electronic unit or the like performs calculation and processing on the electrical signal acquired by the electrode, biological information such as an electrocardiogram, a heart rate, a pulse rate, a breathing rate, a blood pressure, a body temperature, a myopotential, or sweating is obtained, for example.

The garment preferably includes a fastener used for connection to the electronic unit. The fastener is a so-called hook, and examples thereof include a stainless-steel hook. The conductive layer and the electronic unit can be electrically connected to each other via the fastener.

The electronic unit or the like is preferably detachable from the garment. Further, the electronic unit or the like preferably includes display means, storage means, communication means, a USB connector, and the like. The electronic unit or the like may also include, for example, a sensor capable of measuring environmental information such as atmospheric temperature, humidity, or atmospheric pressure, or a sensor capable of measuring positional information using a GPS.

As the element for detecting a positional shift of the body, a known element can be used, and examples of the element include optical displacement sensors, ultrasonic displacement sensors, contact type displacement sensors, and the like.

By using the garment of the present invention, biological information about the wearer can be stably and accurately measured, and the measured biological information is applicable also to a technique of grasping a human psychological or physiological state. For example, the degree of relaxation can be detected for mental training, sleepiness can be detected for preventing drowsy driving, or an electrocardiogram can be measured for diagnosis of depression, stress, or the like.

The present application claims priority based on Japanese Patent Application No. 2020-49716 filed on Mar. 19, 2020. All the contents described in Japanese Patent Application No. 2020-49716 are incorporated herein by reference.

### EXAMPLES

Hereinafter, the present invention is more specifically described by way of examples. The present invention is not limited by the following examples, and can be modified and implemented within the scope of complying with the gist of the descriptions above and below. Those all fall within the technical scope of the present invention. In addition, a part means a mass part.

### Example 1

A garment (Tshirt) shown in FIG. 1 was produced. A covered yarn (hereinafter, sometimes referred to as a covered yarn 1) obtained by winding a 70-denier nylon fiber on the surface of a 40-denier polyurethane fiber was used for the portion at the first clothing fabric I, a spun yarn (hereinafter, sometimes referred to as a spun yarn 2) having a yarn number 15 and obtained by intertwining two yarns each obtained by intertwining two cotton fibers each having a yarn number 60 was used for the portions at the second clothing fabrics 2a and 2b, and two covered yarns 1 each used for the first clothing fabric were aligned for the portions at the third clothing fabrics 3a and 3b, whereby the garment was produced. In the production of the garment, a “WHOLEGARMENT flat-knitting machine” manufactured by SHIMA SEIKI MFG., LTD., was used. Specifically, the portion at the second clothing fabric 2b was knitted in jersey stitch by using the spun yarn 2, then the portion at the first clothing fabric 1 was knitted in jersey stitch by using the covered yarn 1, then the portions at the third clothing fabrics 3a and 3b were knitted in garter stitch by using the aligned covered yarns 1, and then the portion at the second clothing fabric 2a was knitted in jersey stitch by using the spun yarn 2. All of the first clothing fabric, the second clothing fabrics, and the third clothing fabrics were made without sewing.

The third clothing fabrics 3a and 3b were disposed between the first clothing fabric 1 and the second clothing fabric 2a disposed on the upper side in the body length direction relative to the first clothing fabric 1, the third clothing fabrics 3a and 3b being respectively disposed in a region that included the axillary line on the right side and that occupied 17% of the entire circumference around the torso, and a region that included the axillary line on the left side and that occupied 17% of the entire circumference around the torso. Each third clothing fabric was disposed so as to be left-right symmetric with respect to the corresponding axillary line in the circumferential direction around the torso. The third clothing fabric was not disposed in a chest region that was located at the center position in the body width direction of the front body of the garment and that ranged over 33% of the entire circumference around the torso in the body width direction.

The weight per unit area of the obtained first clothing fabric was 218 g/m², and the weight per unit area of each of the obtained second clothing fabrics was 172 g/m². The weight per unit area was measured in accordance with “Mass per unit area in standard state” specified in 8.3.2 of JIS L 1096 (2010).

### Example 2

A first clothing fabric was knitted in jersey stitch with the same covered yarn 1 as that in the example 1 by using a single circular-knitting machine manufactured by Precision Fukuhara Works, Ltd. In addition, second clothing fabrics were knitted in jersey stitch with the same spun yarn 2 as that in the example 1 by using the circular-knitting machine. In addition, the mode of a double circular-knitting machine manufactured by Precision Fukuhara Works, Ltd., was set to a rib gauging mode, and two said covered yarns 1 were aligned, and third clothing fabrics were knitted as draw-off fabrics (in 2x2 rib stitch). The first clothing fabric, the second clothing fabrics, and the third clothing fabrics having been obtained were scoured, dyed, and finished according to dyeing treatment conditions for general circular knitted fabrics. The weight per unit area of the finished first clothing fabric was 205 g/m², the weight per unit area of each of the finished second clothing fabrics was 170 g/m², and the weight per unit area of each of the finished third clothing fabrics was 320 g/m².

Each of the first clothing fabric, the second clothing fabrics, and the third clothing fabrics having been obtained were sewed to respectively obtain the portion at the first clothing fabric 1, the portions at the second clothing fabrics 2a and 2b, and the portions at the third clothing fabrics 3a and 3b corresponding to those in the example 1. Thus, a T-shirt resulting from cutting and sewing was produced. The third clothing fabrics 3a and 3b (draw-off fabrics) were sewed with the horizontal direction of the knitted fabrics being set as the body length direction so as to be elongated in the body length direction to a large extent.

### Comparative Example

A garment (T-shirt) was produced from the covered yarn 1, the spun yarn 2, the yarn obtained by aligning the covered yarns 1 prepared in the above example 1, by using the “WHOLEGARMENT flat-knitting machine” manufactured by SHIMA SEIKI MFG., LTD. Specifically, the second clothing fabric 2b was knitted in jersey stitch by using the spun yarn 2, then the first clothing fabric 1 was knitted in jersey stitch by using covered yarn 1, then the third clothing fabrics were knitted in garter stitch by using the aligned covered yarns 1, and then the second clothing fabric 2a was knitted in jersey stitch by using the spun yarn 2. The third clothing fabrics were formed between the first clothing fabric 1 and either of the second clothing fabrics so as to extend over the entire circumference around the torso. All of the first clothing fabric, the second clothing fabrics, and the third clothing fabrics were made without sewing. The weight per unit area of the obtained first clothing fabric was 218 g/m², and the weight per unit area of each of the obtained second clothing fabrics was 172 g/m².

Next, regarding the first clothing fabric, the second clothing fabrics, and the third clothing fabrics obtained in each of the example 1, the example 2, and the comparative example, the elongation rates thereof in the body length direction were measured. The elongation rates were measured according to method A (constant-speed elongation method) stipulated in 8.16.1 of JIS L 1096. A grip interval for a test piece was set to 3 cm × 3 cm, and a tension speed was set to 30 cm/min.

The elongation rate of the first clothing fabric at a stress of 0.5 N in the body length direction was 15%, and the elongation rate of each of the third clothing fabrics at a stress of 0.5 N in the body length direction was 75%. That is, the elongation rate of the third clothing fabric at a stress of 0.5 N in the body length direction was 500% of the elongation rate of the first clothing fabric at a stress of 0.5 N in the body length direction. The stress of the first clothing fabric upon elongating by 50% in the body width direction was 7.2 N, and the stress of each of the second clothing fabrics upon elongating by 50% in the body width direction was 3.1 N. That is, the stress of the second clothing fabric upon elongating by 50% in the body width direction was 43% of the stress of the first clothing fabric upon elongating by 50% in the body width direction.

Next, a conductive paste for forming an electrode and a wire was produced through the following procedure.

### Conductive Paste

20 parts by mass of nitrile rubber (Nipol (registered trademark) DN003 manufactured by Zeon Corporation) was dissolved in 80 parts by mass of isophorone to prepare an NBR solution. In 100 parts by mass of the obtained NBR solution were blended 110 parts by mass of silver particles (“agglomerate silver powder G35” manufactured by DOWA Electronics Materials Co., Ltd., average particle size: 5.9 µm),and the mixture was kneaded by a triple roll mill to give a conductive paste.

Next, an electrode and a wire were formed by using the obtained conductive paste on the first clothing fabric in each of the example 1, the example 2, and the comparative example under the following conditions, whereby a garment for measuring biological information was produced.

### Electrodes and Wire

The obtained conductive paste was applied onto a release sheet and dried by a hot air dry oven set to 120° C. for 30 minutes or more, to produce a release sheet-attached and sheet-shaped conductive layer. Next, a polyurethane hot-melt sheet was superposed on the conductive layer surface of the obtained release sheet-attached and sheet-shaped conductive layer, and furthermore, a polyethylene film having a thickness of 50 µm was superposed. Then, these layers were pressed and heated by using a hot-press machine under conditions of a pressure of 0.5 kgf/cm², a temperature of 130° C., and a pressing time of 20 seconds, whereby a layered product was obtained. The obtained layered product was cut into a size of a length of 12 cm and a width of 2 cm by using a Thomson blade. Next, the above polyester film-based release sheet was peeled, to obtain a polyurethane hot-melt sheet-attached and sheet-shaped conductive layer.

Next, the obtained polyurethane hot-melt sheet-attached and sheet-shaped conductive layer (having a length of 12 cm and a width of 2 cm) was disposed with the polyethylene film side thereof being oriented to a polyurethane hot-melt sheet separately produced and having a length of 13 cm and a width of 2.4 cm such that the sheet-shaped conductive layer was located at the center of the polyurethane hot-melt sheet (i.e., such that blank spaces each measuring 0.5 cm in the length direction and blank spaces each measuring 0.2 cm in the width direction were formed), and the polyurethane hot-melt sheet was sandwiched by the polyethylene film while being heated and pressed, to be stacked. Consequently, a layered product composed of the polyurethane hot-melt sheet and the sheet-shaped conductive layer was produced. Here, the polyurethane hot-melt sheet corresponds to the above first insulating layer. Next, the same polyurethane hot-melt sheet as that forming the above first insulating layer was stacked, from a portion apart from an end of the sheet-shaped conductive layer by 2 cm, in a region with a length of 5 cm and a width of 2.4 cm so as to cover a portion of the above first insulating layer and a portion of the sheet-shaped conductive layer, whereby a second insulating layer was formed on the portion of the above sheet-shaped conductive layer. That is, a stretchable electrode part in which a device connection portion, an insulating portion, and an electrode were disposed in the longitudinal direction in this order was produced. The device connection portion was located at an end portion of the stretchable electrode part, was an exposed portion of the conductive layer, and had a length of 2 cm and a width of 2 cm. The insulating portion had a stacked structure composed of the first insulating layer, the conductive layer, and the second insulating layer. The electrode was located on the opposite end portion of the stretchable electrode part, was an exposed portion of the conductive layer, and had a length of 5 cm and a width of 2 cm. The exposed portion of the conductive layer as the device connection portion corresponds to the above electrode, and a portion of the conductive layer at the insulating portion corresponds to the above wire. Next, two said stretchable electrode parts produced as described above were attached so as to be left-right symmetric with each other at predetermined positions on the skin-side surface on the inner side of the front body of the first clothing fabric obtained in each of the example 1, the example 2, and the comparative example. That is, the skin-side surface was located on the side on which the electrode surfaces were brought into contact with the skin of a wearer. Consequently, an undershirt for measuring biological information was produced. The number of the electrodes provided on the front body of the clothing fabric was two, the total area of the electrode surfaces of the two electrodes was 22 cm², and the average thickness of each of the electrodes was 90 µm.

Next, garments (T-shirts) each obtained by attaching an electronic unit to the corresponding undershirt were worn by subjects. The subjects were ten males aged from 25 to 52, and had heights of 160 to 175 cm, weights of 58 to 78 kg, shoulder widths of 42 to 47 cm, chest girths of 82 to 93 cm, and waist girths of 72 to 85 cm.

In cases where, in a state where a subject wore any of the garments, the electrodes thereof were disposed within a region having a center at the epigastrium and extending over 3 cm to the upper and lower sides, the garment was evaluated as being “favorable”. Meanwhile, in cases where, in a state where a subject wore any of the garments, the electrodes thereof were disposed outside this region, the garment was evaluated as being “unfavorable”. As a result, for all of the ten subjects, the garments obtained in the example 1 and the example 2 were evaluated as being “favorable” since the electrodes thereof were disposed within the desired region. The garments in the example 1 and the example 2 were easy to wear. On the other hand, for seven subjects out of the ten subjects, the garment in the comparative example was evaluated as being “unfavorable” since the electrodes thereof were disposed outside the desired region. The garment in the comparative example was difficult to wear.

In addition, for each subject wearing any of the garments, evaluations were made as to whether the electrodes thereof were displaced from the desired position when the subject raised and lowered both of his arms. As a result, each subject wearing either of the garments obtained in the example 1 and the example 2 seldom experienced displacement of the electrodes thereof from the desired position even when the subject repetitively raised and lowered both of his arms. On the other hand, each subject wearing the garment in the comparative example occasionally experienced displacement of the electrodes thereof from the desired position as the subject repetitively raised and lowered both of his arms.

1. A garment for measuring biological information having a torso portion, a stretchable conductive member being formed on the garment with an adhesive layer interposed therebetween, wherein
the torso portion comprises at least a first clothing fabric, a second clothing fabric, and a third clothing fabric having elongation rates in a body length direction, the elongation rates being different from one another,
the first clothing fabric is disposed in a circumferential direction around a torso,
the conductive member is formed on the first clothing fabric,
the second clothing fabric is disposed on each of an upper side in the body length direction and a lower side in the body length direction relative to the first clothing fabric,
the third clothing fabric is disposed between the first clothing fabric and the second clothing fabric that is disposed on the upper side in the body length direction relative to the first clothing fabric, the third clothing fabric being disposed in each of
a region that includes an axillary line on a right side and that occupies 5 to 30% of an entire circumference around the torso, and
a region that includes an axillary line on a left side and that occupies 5 to 30% of the entire circumference around the torso, and
the elongation rate in the body length direction of the third clothing fabric is higher than the elongation rate in the body length direction of the first clothing fabric.

2. The garment according to claim 1, wherein
the elongation rate of the third clothing fabric at a stress of 0.5 N in the body length direction is not lower than 200% of the elongation rate of the first clothing fabric at a stress of 0.5 N in the body length direction.

3. The garment according to claim 1, wherein
the third clothing fabric has a bellows shape so as to be stretchable in the body length direction.

4. The garment according to claim 1, wherein
the third clothing fabric is not disposed in a chest region that is located at a center position in a body width direction of a front body of the garment and that occupies 5 to 40% of the entire circumference around the torso.

5. The garment according to claim 1, wherein
each third clothing fabric is disposed so as to be left-right symmetric with respect to the corresponding axillary line in the circumferential direction around the torso.

6. The garment according to claim 1, wherein
a stress of the second clothing fabric upon elongating by 50% in a body width direction is not higher than 70% of a stress of the first clothing fabric upon elongating by 50% in the body width direction.

7. The garment according to claim 1, wherein
each of the first clothing fabric, the second clothing fabric, and the third clothing fabric is a knitted fabric.

8. The garment according to claim 1, wherein
the garment is a knitted fabric made without sewing.

9. The garment according to claim 1, wherein
the conductive member is an electrode configured to detect an electrical signal from a body and/or an element configured to detect a positional shift of the body.

10. The garment according to claim 9, wherein
the electrode is configured to detect electrocardiographic potential or myopotential.

11. The garment according to claim 1, wherein
the garment is configured to cover a chest.
